(19) United States
(12) Patent Application Publication
(10) Pub. No.: US 2026/0034037 A1
(43) Pub. Date: Feb. 5, 2026

(54) STABILIZED AVOBENZONE IN COSMETIC SUNSCREEN COMPOSITIONS

(71) Applicant: BEIERSDORF AG, Hamburg (DE)

(72) Inventors: Donna HWANG, Leonia, NJ (US); Yongyi Zhang, New Providence, NJ (US); Eduardo Ruvolo, Flanders, NJ (US)

(21) Appl. No.: 18/790,788

(22) Filed: Jul. 31, 2024

(51) Int. Cl. A61K 8/35; A61K 8/37; A61K 8/92; A61Q 17/04

(52) U.S. Cl. CPC A61K 8/35; A61K 8/37; A61K 8/922; A61Q 17/04; A61K 2800/30

(57) ABSTRACT

A cosmetic sunscreen composition comprises avobenzone and one or more of Raspberry Ketone, γ-Oryzanol and Ethyl Ferulate to stabilize the avobenzone.

### BACKGROUND OF THE INVENTION

### 1. Field of the Invention

The present invention relates to a cosmetic sunscreen composition comprising a combination of 4-(tert-butyl)-4′-methoxydibenzoylmethane (avobenzone) and one or more of Raspberry Ketone, γ-Oryzanol and Ethyl Ferulate, as well as the use of one or more Raspberry Ketone, γ-Oryzanol and Ethyl Ferulate for enhancing the photostability of avobenzone in a cosmetic sunscreen composition.

### 2. Discussion of Background Information

Organic UV filters are required to absorb solar radiation energy and then quickly release it in order to readily absorb additional solar energy. However, some UV filters can be photodegraded over time in the sun, losing their absorption capability and thus, their ability to protect skin from sun damage.

An example of such an UV filter is 4-(tert-butyl)-4′-methoxydibenzoylmethane (avobenzone, INCI: Butyl Methoxydibenzoylmethane), which is a globally approved UVA filter. Currently avobenzone is the only UVA sunscreen approved by the U.S. Food & Drug Administration (FDA). While avobenzone provides very good coverage in the UVA region of the electromagnetic spectrum, it has instability issues because it degrades during exposure to UV radiation and loses its ability to absorb the radiation in the UVA range.

Extensive studies have been carried out to investigate the photostability of avobenzone with different UV filters.

The stabilization of avobenzone by octocrylene (another UV filter) is known. Recently, however, octocrylene has been linked to aquatic toxicity, with the potential to harm coral health. Further, some experts believe that octocrylene is a potentially harmful ingredient. Regardless of whether all these concerns are justified or not, creating octocrylene-free cosmetics has become an urgent goal.

Some studies indicate that another UV filter-bis-ethylhexyloxyphenol methoxyphenyl triazine (Bemotrizinol)-can stabilize avobenzone and prevent avobenzone from photodegradation. But Bemotrizinol is not an FDA approved filter and thus, it is not allowed to be marketed and used in the US.

In addition, there have been considerable efforts to stabilize avobenzone by combining it with photostabilizers including diethylhexyl syringylidene malonate (trade name Oxynex), trimethoxybenzylidene pentanedione (trade name: Synoxyl HSS), and ethylhexyl methoxycrylene (trade name: SolaStay S1). These photostabilizers are synthetic materials that do not meet the global sustainable development goals. Thus, finding a sustainable photostabilizer to stabilize avobenzone is challenging.

### SUMMARY OF THE INVENTION

The present invention provides a cosmetic sunscreen composition which comprises a combination of

(a) 4-(tert-butyl)-4′-methoxydibenzoylmethane (avobenzone), and

(b) one or more of Raspberry Ketone, γ-Oryzanol, and Ethyl Ferulate.

It was unexpected for those of skill in the art that these natural substances (Raspberry Ketone, γ-Oryzanol and Ethyl Ferulate) can significantly photo-stabilize avobenzone.

Accordingly, the present invention also provides a method of enhancing the photostabilization of avobenzone in a cosmetic sunscreen composition. The method comprises incorporating into the cosmetic sunscreen composition one or more of Raspberry Ketone, γ-Oryzanol and Ethyl

Ferulate.

The use of one or more of Raspberry Ketone, γ-Oryzanol and Ethyl Ferulate for enhancing the photostability of avobenzone in a sunscreen composition is also provided by the present invention.

In the context of the present disclosure, formulations like “according to the invention”, “preparation according to the invention” etc. always refer to the compositions, processes and uses according to the invention.

It is preferred according to the invention if the cosmetic sunscreen composition comprises avobenzone in a concentration of from about 0.01% to about 5.0% by weight, e.g., from about 0.1% to about 5.0% by weight, based on the total weight of the composition.

If the sunscreen composition according to the invention comprises γ-Oryzanol this substance is preferably present in a concentration of from about 0.1% to about 5.0% by weight, based on the total weight of the composition.

If the sunscreen composition according to the invention comprises Ethyl Ferulate this substance is preferably present in a concentration of from about 0.01% to about 5.0% by weight, e.g., from about 0.1% to about 5.0% by weight, based on the total weight of the composition.

If the sunscreen composition according to the invention comprises Raspberry Ketone this substance is preferably present in a concentration of from about 0.1% to about 5.0% by weight, based on the total weight of the composition.

It further is preferred for the sunscreen composition of the invention to additionally comprise isopropyl lauroyl sarcosinate. In this case this substance is preferably present in a concentration of from about 1% to about 15% by weight, based on the total weight of the composition.

The sunscreen composition according to invention may further comprise one or more other substances such as, e.g., further lipids, especially one or more of the following sustainable substances: dibutyl adipate, isoamyl laurate, isoamyl cocoate, phenoxyethyl caprylate, caprylic triglyceride, capric triglyceride, dicaprylyl carbonate, propylheptyl caprylate, propylene glycol dicaprylate/dicaprate, propylene glycol diheptanoate, ethyl PG-acetal levuinate, coco glycerides, fatty alcohols, triglycerides, esters, and any combination thereof.

The sunscreen composition of the invention will often comprise one or more (e.g., 1, 2, 3, 4 or 5) additional UV filter substances such as, e.g., homosalate and/or ethylhexyl salicylate. However, the composition is preferably free of octocrylene.

It is also preferred for the composition to be free of 3-(4-methylbenzylidene)camphor and 2-hydroxy-4-methoxybenzophenone (oxybenzone). Propyl paraben and butyl paraben, 3-iodo-2-propynyl butylcarbamate are also preferably absent from the composition.

If the composition of the invention comprises homosalate this substance is preferably present in a concentration of from about 10% to about 20% by weight, based on the total weight of the composition.

If the composition of the invention comprises ethylhexyl salicylate this substance is preferably present in a concentration of from about 3% to about 8% by weight, based on the total weight of the composition.

The composition of the invention may further comprise xanthan gum. In this case this substance is preferably present in a concentration of from about 0.1% to about 0.8% by weight, based on the total weight of the composition.

The composition of the invention may further comprise phenoxyethanol. In this case this substance is preferably present in a concentration of from about 0.5% to about 1.3% by weight, based on the total weight of the composition.

The composition of the invention may further comprise ethylhexylglycerin. In this case this substance is preferably present in a concentration of from about 0.1% to about 0.5% by weight, based on the total weight of the composition.

Preferred embodiments of the invention include a composition which further comprise one or both of Rosmarinus Officinalis (Rosmary) Leaf Extract and Haematococcus Pluvialis Extract.

A preferred composition according to the invention further comprises Helianthus Annuus (Sunflower) Seed Oil, preferably in a concentration of from about 1% to about 10% by weight, based on the total weight of the composition.

It also is preferred for the composition of the invention to further comprise tocopherol and/or tocopheryl acetate.

The composition of the invention may further comprise one or more additional stabilizers for avobenzone. Such stabilizers preferably include diethylhexyl syringylidenemalonate.

The composition of the invention may be present in various forms such as, e.g., in the form of a lotion, a cream, an ointment, an emulsion, a gel, etc.

It is preferred for the composition of the invention to be present in the form of an emulsion. In such a case the preferred system is an oil-in-water emulsion (O/W emulsion).

The composition of the invention may further comprise all of the substances which are conventionally used in cosmetic compositions such as, e.g., moisturizers, emollients, lubricants, thickeners, waxy solids, emulsifiers, film forming polymers, and other common cosmetic formulation additives. The composition may also include active ingredients which can provide skin benefits such as, e.g., niacinamide, vitamin C, retinol, thiamidol, ubiquinone, acetyl hydroxyproline, hyaluronic acid, bakuchiol, ceramides, panthenol, and any combination thereof.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description making apparent to those of skill in the art how the several forms of the present invention may be embodied in practice.

Table I lists the ingredients and their concentrations used for four exemplary compositions: Exp1, Exp2, Exp3, and Exp4. The process of making these compositions comprises heating phase A and phase B separately to 80° C. Once at this temperature, the two phases are combined using a high-shear mixer to form a pre-emulsion. The pre-emulsion is further homogenized to form a uniform emulsion.

TABLE I
Formulation examples

| | | Example formula | | | |
|---|---|---|---|---|---|
| | | (% by weight) | | | |
| Phase | INCI Name | Exp1 | Exp2 | Exp3 | Exp4 |
| A | Homosalate | 15.0 | 15.0 | 15.0 | 15.0 |
| A | Ethylhexyl Salicylate | 5.0 | 5.0 | 5.0 | 5.0 |
| A | Butyl Methoxydibenzoylmethane | 3.0 | 3.0 | 3.0 | 3.0 |
| A | C12-15 Alkyl benzoate | 10.0 | — | — | — |
| A | Isopropyl Lauroyl | — | 10.0 | 10.0 | 10.0 |
| | Sarcosinate | | | | |
| A | Diethylhexyl | — | — | 2.0 | — |
| | Syringylidenemalonate | | | | |
| A | Oryzanol | — | — | — | 3.0 |
| B | Xanthan Gum | 0.5 | 0.5 | 0.5 | 0.5 |
| B | Glycerin | 2.0 | 2.0 | 2.0 | 2.0 |
| B | Sodium Stearoyl Glutamate | 1.0 | 1.0 | 1.0 | 1.0 |
| B | Phenoxyethanol | 0.9 | 0.9 | 0.9 | 0.9 |
| B | Ethylhexylglycerin | 0.2 | 0.2 | 0.2 | 0.2 |
| B | Citric Acid | 0.05 | 0.05 | 0.05 | 0.05 |
| B | Water | Qs. | Qs. | Qs. | Qs. |
| | | To 100 | To 100 | To 100 | To 100 |

The formulation performance is evaluated by comparing the UV absorbance measurements of the samples before and after irradiation. To obtain these measurements, each sample is evenly applied at 1.2 mg/cm² to the roughened side of a polymethylmethacrylate (PMMA) plate. Then, the plate is allowed to equilibrate for 15 minutes in the dark. The plate is measured using UV-2000 Ultraviolet Transmittance Analyzer by Labsphere Inc. to determine the transmittance values before the irradiation. The transmittance values are further converted into mean absorbance values. After the measurement, the sample is irradiated with a Solar Light Company model LS1000-8R-UV solar simulator with a light source that can produce a continuous spectral distribution of UV radiation from 290 to 400 nanometers. The average intensity of the solar simulator as measured is 4 mW/cm², equivalent to m Joule/second/cm². The sample treated plate was then exposed for the prescribed time to achieve the UV exposure dosage for each individual plate listed in Table II. A full day of summer Sun exposure is approximately equivalent to 21 m Joules/cm². This supports the relevance of the chosen dosages in the present invention. After irradiation, each plate is measured again using the Labsphere analyzer to obtain the transmittance values, which are then converted to mean absorbance values.

Avobenzone has a maximum absorbance intensity at 357 nm, which is used to calculate the avobenzone retention percent values for each formulation. Table II shows the percent retention of avobenzone after different irradiation dosages. The sustainable solvent by itself as described in Exp2 helps to prevent the breakdown of avobenzone when the formula is irradiated at low dosage compared to Exp1, but it has limited protection to prevent avobenzone from breakdown under higher irradiation dosage. The composition of Exp4 as described in the present invention demonstrates a strong retention of avobenzone and the performance of the invention composition with a sustainable photostabilizer has a stronger photoprotection than the synthetic one (Exp3) as shown in Table II.

TABLE II
Percent retention of Avobenzone in example
formulations listed in Table I

| Irradiation Dose | Exp1 | Exp2 | Exp3 | Exp4 |
|---|---|---|---|---|
| 0 m Joule/cm² | 100.0% | 100.0% | 100.0% | 100.0% |
| 10 m Joule/cm² | 56.5% | 87.3% | 96.2% | 95.7% |
| 20 m Joule/cm² | 22.1% | 72.1% | 89.6% | 91.7% |
| 35 m Joule/cm² | 6.7% | 46.6% | 76.3% | 81.3% |

Additional examples provided in Table III for the composition of sustainable photostabilizers, especially the carotenoids and terpenoids type of materials, include Rosmarinus Officinalis (Rosemary) Leaf Extract and Haematococcus Pluvialis extract. Examples of the composition of sustainable photostabilizers, especially the phenol or polyphenol type of materials, include ethyl ferulate and raspberry ketone, and any combination thereof also described in Table III, which include four example formulations: Exp5. Exp6. Exp7. and Exp8. The process of making these formulations in Table III is the same as that described above for Table I.

TABLE III
Formulation examples

| | | Example formula | | | |
|---|---|---|---|---|---|
| | | (% by weight) | | | |
| Phase | INCI Name | Exp5 | Exp6 | Exp7 | Exp8 |
| A | Homosalate | 15.0 | 15.0 | 15.0 | 15.0 |
| A | Ethylhexyl Salicylate | 5.0 | 5.0 | 5.0 | 5.0 |
| A | Butyl Methoxydibenzoylmethane | 3.0 | 3.0 | 3.0 | 3.0 |
| A | Polyester-8 | 3.0 | 3.0 | 3.0 | 3.0 |
| A | Isopropyl Lauroyl Sarcosinate | — | 10.0 | 10.0 | 10.0 |
| A | Helianthus Annuus (Sunflower) | — | 5.58 | — | — |
| | Seed Oil | | | | |
| A | Simmondsia Chinensis (Jojoba) | — | — | 1.0 | — |
| | Seed Oil | | | | |
| A | Ethyl Ferulate (and) Rosmarinus | — | 0.42 | — | — |
| | Officinalis (Rosemary) | | | | |
| | Leaf Extract | | | | |
| B | Haematococcus Pluvialis | — | — | 1.0 | — |
| | Extract | | | | |
| B | Raspberry ketone | — | — | — | 2.0 |
| B | Xanthan Gum | 0.5 | 0.5 | 0.5 | 0.5 |
| B | Glycerin | 2.0 | 2.0 | 2.0 | 2.0 |
| B | Sodium Stearoyl Glutamate | 1.0 | 1.0 | 1.0 | 1.0 |
| B | Phenoxyethanol | 0.9 | 0.9 | 0.9 | 0.9 |
| B | Ethylhexylglycerin | 0.2 | 0.2 | 0.2 | 0.2 |
| B | Citric Acid | 0.05 | 0.05 | 0.05 | 0.05 |
| B | Water | Qs. | Qs. | Qs. | Qs. |
| | | To 100 | To 100 | To 100 | To 100 |

The formulation performance of Table IV is evaluated the same way as described for Table II. As shown in Table IV, the compositions of the present invention have a better retention of avobenzone and prevent avobenzone from irradiation breakdowns especially in the higher irradiation dosages.

TABLE IV
Percent retention of Avobenzone in example
formulations listed in Table III

| Energy Applied | Exp5 | Exp6 | Exp7 | Exp8 |
|---|---|---|---|---|
| 0 m Joule/cm² | 100.0% | 100.0% | 100.0% | 100.0% |
| 10 m Joule/cm² | 91.8% | 94.3% | 96.8% | 95.0% |
| 20 m Joule/cm² | 81.4% | 89.5% | 90.2% | 89.1% |
| 35 m Joule/cm² | 62.3% | 80.3% | 80.0% | 80.7% |

Adding more information and fixing, Table V lists more examples for the composition of sustainable photostabilizers, which include four example formulations: Exp9, Exp10, Exp11, and Exp12. The process of these formulations in Table V is the same as that described above for Table I.

TABLE V
Formulation examples

| | | Example formula | | | |
|---|---|---|---|---|---|
| | | (% by weight) | | | |
| Phase | INCI Name | Exp9 | Exp10 | Exp11 | Exp12 |
| A | Homosalate | 15.0 | 15.0 | 15.0 | 15.0 |
| A | Ethylhexyl Salicylate | 5.0 | 5.0 | 5.0 | 5.0 |
| A | Butyl Methoxydibenzoylmethane | 3.0 | 3.0 | 3.0 | 3.0 |
| A | Isopropyl Lauroyl Sarcosinate | 10.0 | 10.0 | 10.0 | 10.0 |
| A | Helianthus Annuus (Sunflower) | 8.0 | — | 6.0 | — |
| | Seed Oil, Ethyl Ferulate, | | | | |
| | Rosmarinus Officinalis | | | | |
| | (Rosemary) Leaf Extract, | | | | |
| | Tocopherol | | | | |
| A | Simmondsia Chinensis (Jojoba) | — | 4.0 | — | — |
| | Seed Oil, Haematococcus Pluvialis | | | | |
| | Extract, Tocopherol, Helianthus | | | | |
| | Annuus (Sunflower) Seed Oil | | | | |
| B | Raspberry ketone | — | — | 2.0 | 4.0 |
| B | Xanthan Gum | 0.5 | 0.5 | 0.5 | 0.5 |
| B | Glycerin | 2.0 | 2.0 | 2.0 | 2.0 |
| B | Sodium Stearoyl Glutamate | 1.0 | 1.0 | 1.0 | 1.0 |
| B | Phenoxyethanol | 0.9 | 0.9 | 0.9 | 0.9 |
| B | Ethylhexylglycerin | 0.2 | 0.2 | 0.2 | 0.2 |
| B | Citric Acid | 0.05 | 0.05 | 0.05 | 0.05 |
| B | Water | Qs. | Qs. | Qs. | Qs. |
| | | To 100 | To 100 | To 100 | To 100 |

Adding more information and fixing, the formulation performance of Table VI is evaluated the same way as described for Table IL. As shown in Table VI, the present invention composition especially Exp12 with raspberry ketone has a better retention of avobenzone and prevents avobenzone from irradiation breakdowns especially in the higher irradiation dosages.

TABLE VI
Percent retention of Avobenzone in example
formulations listed in Table V

| Irradiation Dose | Exp9 | Exp10 | Exp11 | Exp12 |
|---|---|---|---|---|
| 0 m Joule/cm² | 100% | 100% | 100% | 100% |
| 10 m Joule/cm² | 94% | 96% | 93% | 96% |
| 20 m Joule/cm² | 86% | 86% | 87% | 91% |
| 35 m Joule/cm² | 72% | 70% | 76% | 84% |

The absorbance intensity data at 357 nm are used to calculate the UV absorbance retention percentage values

What is claimed is:

1. A cosmetic sunscreen composition, wherein the composition comprises
(a) 4-(tert-butyl)-4′-methoxydibenzoylmethane (avobenzone), and
(b) one or more of Raspberry Ketone, γ-Oryzanol, and Ethyl Ferulate.

2. The composition of claim 1, wherein the composition comprises at least Raspberry Ketone.

3. The composition of claim 1, wherein the composition comprises at least γ-Oryzanol.

4. The composition of claim 1, wherein the composition comprises at least Ethyl Ferulate.

5. The composition of claim 1, wherein the composition comprises avobenzone in a concentration of from 0.01% to 5% by weight, based on a total weight of the composition.

6. The composition of claim 3, wherein the composition comprises γ-Oryzanol in a concentration of from 0.1% to 5% by weight, based on a total weight of the composition.

7. The composition of claim 4, wherein the composition comprises Ethyl Ferulate in a concentration of from 0.01% to 5% by weight, based on a total weight of the composition.

8. The composition of claim 2, wherein the composition comprises Raspberry Ketone in a concentration of from 0.1% to 5% by weight, based on a total weight of the composition.

9. The composition of claim 1, wherein the composition further comprises isopropyl lauroyl sarcosinate.

10. The composition of claim 9, wherein the composition comprises isopropyl lauroyl sarcosinate in a concentration of from 1% to 15% by weight, based on a total weight of the composition.

11. The composition of claim 1, wherein the composition is free of octocrylene.

12. The composition of claim 1, wherein the composition is free of 3-(4-methylbenzylidene)camphor, 2-hydroxy-4-methoxybenzophenone (oxybenzone), propyl paraben, butyl paraben, 3-iodo-2-propynyl butylcarbamate.

13. The composition of claim 1, wherein the composition further comprises homosalate and/or ethylhexyl salicylate.

14. The composition of claim 1, wherein the composition further comprises xanthan gum.

15. The composition of claim 1, wherein the composition further comprises phenoxyethanol and/or ethylhexylglycerin.

16. The composition of claim 1, wherein the composition further comprises Rosmarinus Officinalis (Rosmary) Leaf Extract and/or Haematococcus Pluvialis Extract.

17. The composition of claim 1, wherein the composition further comprises Helianthus Annuus (Sunflower) Seed Oil.

18. The composition of claim 1, wherein the composition further comprises diethylhexyl syringylidenemalonate.

19. The composition of claim 1, wherein the composition further comprises tocopherol and/or tocopheryl acetate.

20. A method of stabilizing avobenzone in a cosmetic sunscreen composition, wherein the method comprises incorporating in the composition one or more of Raspberry Ketone, γ-Oryzanol, and Ethyl Ferulate.
